(19) United States
(12) Patent Application Publication
(10) Pub. No.: US 2016/0324795 A1
(43) Pub. Date: Nov. 10, 2016

(54) SILIBININ NANOPARTICLE AND A METHOD OF TREATING HEPATITIS C THEREOF

(71) Applicant: Kaohsiung Medical University, Kaohsiung (TW)

(72) Inventors: Chun-Ching LIN, Kaohsiung (TW); Liang-Tzung LIN, Kaohsiung (TW); Christopher D. RICHARDSON, Kaohsiung (TW); Wen-Chan HSU, Kaohsiung (TW)

(21) Appl. No.: 15/109,609

(22) PCT Filed: Jan. 27, 2014
(86) PCT No.: PCT/US2014/013134

§ 371 Date: Jul. 1, 2016

(51) Int. Cl. A61K 9/51; A61K 31/357

(52) U.S. Cl. CPC A61K 9/5138; A61K 31/357; A61K 9/5192

(57) ABSTRACT

The present invention discloses a silibinin nanoparticle comprising the compound silibinin and a hydrophilic polymer. Moreover, the silibinin nanoparticle is in form of spherical structure with a particle size of 50 to 200 nm. The present invention also discloses a use of the silibinin nanoparticle to suppress hepatitis C virus infection and a method of treating hepatitis C, by administering the silibinin nanoparticle to a subject in need.

### CROSS REFERENCE TO RELATED APPLICATIONS

The application claims the benefit of International application serial No. PCT/US2014/013134, filed Jan. 27, 2014, the subject matter of which is incorporated herein by reference.

### BACKGROUND OF THE INVENTION

1. Field of the Invention

The present invention generally relates to a silibinin nanoparticle and, more particularly, to a silibinin nanoparticle with improved water solubility. The present invention further relates to a method of treating hepatitis C thereof.

2. Description of the Related Art

As shown in FIG. 1, conventional silibinin, an active ingredient of milk thistle (Silybum marianum (L.) Gaertn.), has hepatoprotective properties that protect liver cells against toxins. In particular, silibinin shows potential for treating hepatitis C in clinical studies. However, the conventional silibinin has poor water solubility and thus has problems of unstable releases, resulting in unstable hepatoprotective effects when administered to a subject. Besides, although silibinin has an improved solubility when dissolved in dimethyl sulfoxide (DMSO), DMSO is dangerous to the human body and therefore silibinin dissolved in DMSO is unusable in clinic.

A conventional silibinin chemical derivative, Legalon®SIL, has an improved water solubility. However, the conventional silibinin chemical derivative is not convenient for administration because it cannot be administered orally.

In light of the above, it is necessary to improve the conventional silibinin.

### SUMMARY OF THE INVENTION

It is therefore the objective of this invention to provide a silibinin nanoparticle in the form of a spherical structure with a particle size of 50 to 200 nm with improved water solubility.

It is another objective of this invention to provide a silibinin nanoparticle with improved hydrophilicity and blood solubility, increasing the bioavailability thereof.

It is yet another objective of this invention to provide a silibinin nanoparticle which can be orally administered to a subject in need, offering a patient-friendly form with regards to ease of administration and convenience.

It is also another objective of this invention to provide a method of treating hepatitis C, by administering the silibinin nanoparticle with improved hydrophilicity, blood solubility, and bioavailability, thereby effectively inhibiting hepatitis C virus infection.

One embodiment of the invention discloses a silibinin nanoparticle comprising the compound silibinin 3,5,7-trihydroxy-2-[3-(4-hydroxy-3-methoxyphenyl)-2-(hydroxymethyl)-2,3-dihydro-1,4-benzodioxin-6-yl]-2,3-dihydrochromen-4-one and a hydrophilic polymer, wherein the silibinin nanoparticle is in the form of a spherical structure with a particle size of 50 to 200 nm.

In a preferred form shown, the hydrophilic polymer is selected from, but not limited to, poloxamer, polyvinyl alcohol, or polyvinylpyrrolidone.

In a preferred form shown, a weight ratio of silibinin and the hydrophilic polymer is between 1:1 and 1:6.

In a preferred form shown, the silibinin nanoparticle is manufactured as follows: dissolving silibinin in ethanol to obtain a silibinin solution; dissolving the hydrophilic polymer in water to obtain a hydrophilic polymer solution; and performing a nanolization reaction between the silibinin solution and the hydrophilic polymer solution to form the silibinin nanoparticle.

The other embodiment of the invention describes a use of the silibinin nanoparticle to suppress hepatitis C virus infection and a method of treating hepatitis C, by administering the silibinin nanoparticle to a subject in need to treat hepatitis C thereof.

In another preferred form shown, the silibinin nanoparticle is orally administered to the subject in need.

In another preferred form shown, the silibinin nanoparticle is administered in a dosage of 5 to 10 mg per kilogram of the subject in need for 2 weeks.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 depicts the chemical structure of silibinin.

FIG. 2 depicts a TEM image of the silibinin nanoparticle of the invention in trial (A).

FIG. 3 depicts XRD patterns of groups B0 to B3 in trial (B).

FIG. 4 depicts DSC curves of groups B0 to B3 in trial (B).

FIG. 5 depicts solubility of groups C1 to C3 in trial (C).

FIG. 6A depicts survival rates of groups D0 to D2 in trial (D).

FIG. 6B depicts body weights of groups D0 to D2 in trial (D).

FIG. 7A depicts serum silibinin concentration of groups E1 and E2 in trial (E).

FIG. 7B depicts tissue silibinin concentration of groups E1 and E2 in trial (E).

FIG. 8 depicts HCVcc infectivity of groups F1 to F7 in trial (F).

In the various figures of the drawings, the same numerals designate the same or similar parts. Furthermore, when the terms “first”, “second”, “third”, “fourth”, “inner”, “outer”, “top”, “bottom” and similar terms are used hereinafter, it should be understood that these terms refer only to the structure shown in the drawings as it would appear to a person viewing the drawings, and are utilized only to facilitate describing the invention.

### DETAILED DESCRIPTION OF THE INVENTION

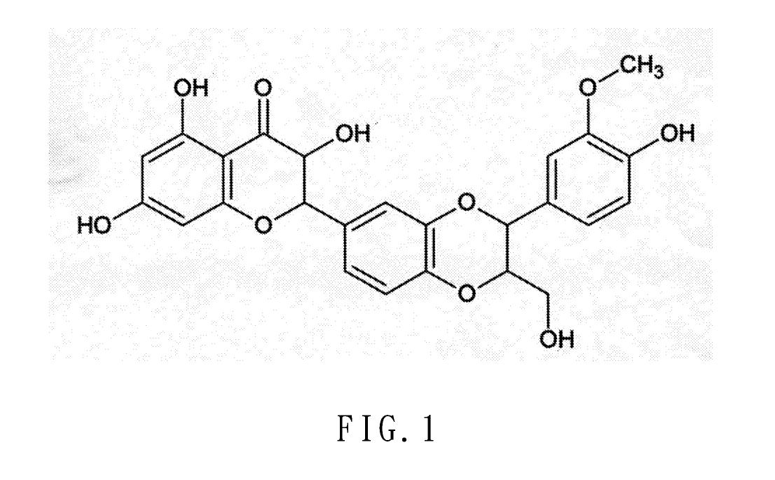

A silibinin nanoparticle according to preferred teachings of the invention comprises: silibinin and a hydrophilic polymer in the form of a spherical structure with a particle size of 50 to 200 nm. As shown in FIG. 1, silibinin is also named as 3,5,7-trihydroxy-2-[3-(4-hydroxy-3-methoxyphenyl)-2-(hydroxymethyl)-2,3-dihydro-1,4-benzodioxin-6-yl]-2,3-dihydrochromen-4-one. The hydrophilic polymer is selected from, but not limited to, poloxamer, polyvinyl alcohol (PVA), or polyvinylpyrrolidone (PVP). Moreover, a weight ratio of silibinin and the hydrophilic polymer is between 1:2 and 1:6.

In a preferred embodiment, the hydrophilic polymer is PVP with a weight ratio of silibinin and PVP of 1:1. Accordingly, the formed silibinin nanoparticle is the spherical structure with a particle size of 50 to 200 nm.

The silibinin nanoparticle can be manufactured as follows: A silibinin solution with a concentration of 2 mg/mL is prepared by dissolving silibinin in ethanol. A hydrophilic polymer solution with a concentration of 0.5 mg/mL is prepared by dissolving PVP in water. The silibinin solution and the hydrophilic polymer solution are mixed in a volumetric ratio of 1:4, followed by performing a nanolization reaction by ultrasonic vibration for 30 minutes (strength of 15 to 30 kHz). Preferably, to prevent silibinin from losing activity caused by heat, the mixed silibinin solution and hydrophilic polymer solution can be cooled down with an ice-water immersion during the nanolization reaction. After the nanolization reaction, ethanol and water are removed, and precipitates are filtrated by a qualitative filter paper.

The silibinin nanoparticle of the invention is preferably kept in a refrigerator in the form of a solution. Alternatively, the silibinin nanoparticle of the invention can be kept in a freezer in the form of a lyophilized powder. In the latter circumstance, the silibinin nanoparticle of the invention can be dissolved in water for further use.

According to the procedure mentioned above, the silibinin nanoparticle of the invention in the form of a spherical structure with a particle size of 50 to 200 nm can be manufactured. The specific surface area is particularly important for activities of the nanoparticle. Via the nanolization reaction, the silibinin nanoparticle of the invention has an improved specific surface area, a total surface area per unit of volume, and therefore has an improved activity, that is, the rate at which the reaction will proceed. Moreover, the hydrophilic polymer has effects on dispersion and combination, thereby not only preventing silibinin from precipitating but also improving hydrophilicity and blood solubility of the silibinin nanoparticle of the invention. Besides, the hydrophilic polymer can result in phase change of silibinin, from crystalline phase to noncrystalline phase, which will be discussed below.

Trial (A). Particle Size of the Silibinin Nanoparticle of the Invention

First, a photon correlation spectroscopy (PCS) is used to analyze the particle size of the silibinin nanoparticle of the invention and reveals the particle size is 166.1±5.5 nm. The result also demonstrates that the yield is 87.9±0.80% and the encapsulation rate is 97.5±0.01%, which means that the silibinin shows high integration with the hydrophilic polymer, PVP.

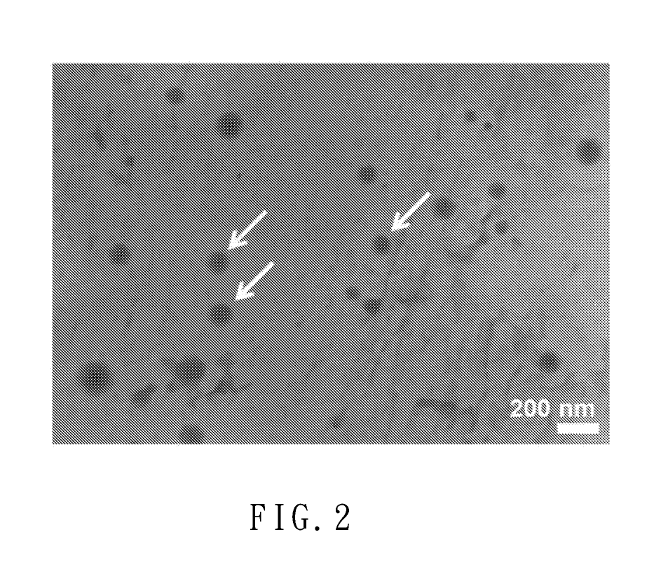

FIG. 2 shows a transmission electron microscopy (TEM) image of the silibinin nanoparticle of the invention. The silibinin nanoparticle of the invention has a particle size of nearly 150 nm, which is similar to the PCS data.

Trial (B). Phase Change of the Silibinin Nanoparticle of the Invention

Samples used in trial (B) are shown in TABLE 1. Group B0 is the hydrophilic polymer solution with the hydrophilic polymer PVP, group B1 is the silibinin solution, group B2 is the mixed silibinin solution and hydrophilic polymer solution (before the nanolization reaction) and group B3 is the mixed silibinin solution and hydrophilic polymer solution (after the nanolization reaction). All of the samples of groups B0 to B3 are in the form of lyophilized powder for the following analyses.

TABLE 1

| | Samples | | |
|---|---|---|---|
| Groups | Silibinin Sol. | PVP Sol. | Nanolization |
| B0 | − | + | − |
| B1 | + | − | − |
| B2 | + | + | − |
| B3 | + | + | + |

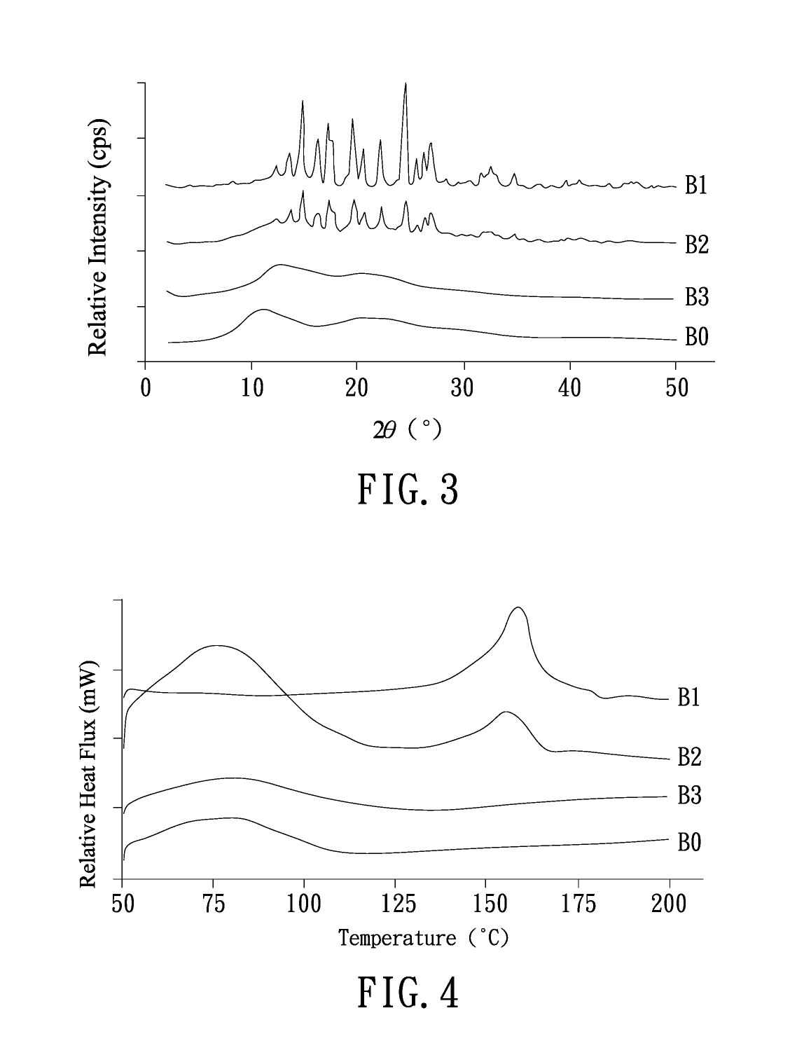

With respect to FIG. 3, groups B1 and B2 show signal at 12 to 28° while group B3 shows no signal at the same position (12 to 28°). That is, no phase change occurs from the mixing of the silibinin solution and the hydrophilic polymer solution, while the nanolization reaction results in the phase change from the crystalline phase to the noncrystalline phase.

Moreover, with reference to FIG. 4, groups B1 and B2 show signal at 155 to 160° C. while group B3 shows no signal at the same temperature (155 to 160° C.). The result further confirms that the nanolization reaction results in the phase change from the crystalline phase to the noncrystalline phase in group B3.

Trial (C). Improved Blood Solubility of the Silibinin Nanoparticle of the Invention

Samples and pH values used in trial (C) are shown in TABLE 2. A solution with pH 7.4 mimics the environment of the blood and a solution with pH 1.2 mimics the environment of the stomach.

TABLE 2

| Groups | Samples | Environment (pH) |
|---|---|---|
| C1 | Silibinin | Blood (pH 7.4) |
| C2 | Silibinin Nanoparticle | Stomach (pH 1.2) |
| C3 | Silibinin Nanoparticle | Blood (pH 7.4) |

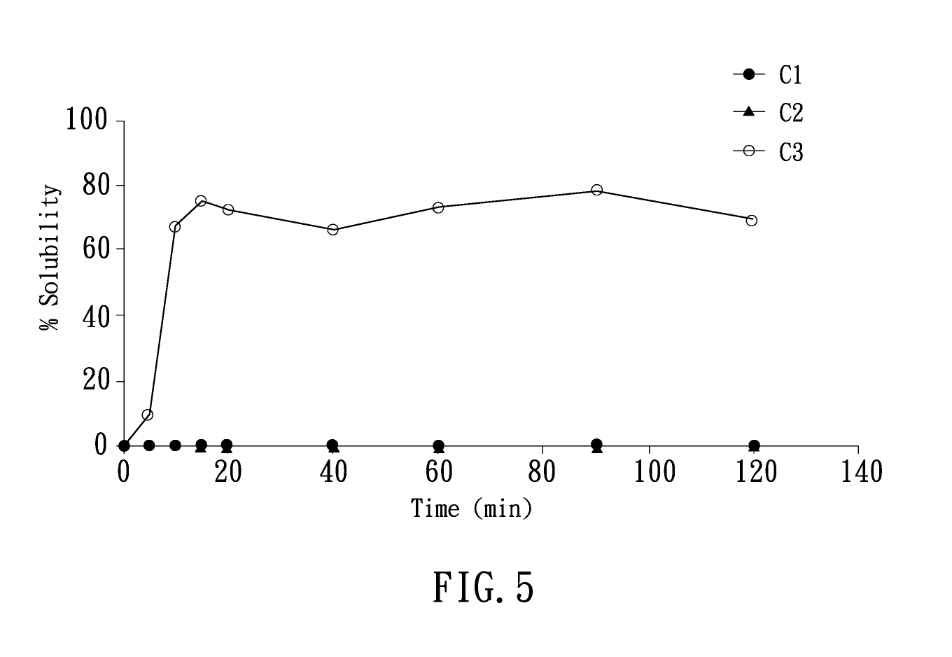

Referring to FIG. 5, the conventional silibinin (group C1) cannot dissolve in the solution mimicking the environment of the blood even after 120 minutes. In contrast, although the silibinin nanoparticle of the invention cannot dissolve in the solution mimicking the environment of stomach (group C2), the silibinin nanoparticle of the invention can effectively dissolve in the solution mimicking the environment of the blood (group C3, a blood solubility of 10% after 5 minutes and a blood solubility of 70% after 10 minutes). That is, compared with the conventional silibinin, the silibinin nanoparticle of the invention has improved blood solubility. The silibinin nanoparticle cannot dissolve in the gastric acid (the environment of the stomach) and therefore protects the active ingredient (silibinin) of the silibinin nanoparticle from being destroyed by the gastric acid.

To evaluate whether the silibinin nanoparticle of the invention is safe to use on a subject, and to further understand the pharmacokinetics of the silibinin nanoparticle of the invention, the following trials (D) and (E) are performed.

Trial (D). Safety Test of the Silibinin Nanoparticle of the Invention

Sprague-Dawley (SD) male rats (6 week-old) purchased from BioLASCO Taiwan Co., Ltd. are used in trials (D) and (E). The SD rats are housed on a 12-hours light and 12-hours dark cycle in an animal room kept at a constant temperature of 25±1° C. and constant humidity of 55±5%. The SD rats are housed for several days before being administered the silibinin nanoparticle of the invention via gastrostomy in a dosage shown in TABLE 3.

TABLE 3

| Groups | Dosage (mg/kg body weight) |
|---|---|
| D0 | — |
| D1 | 5 |
| D2 | 10 |

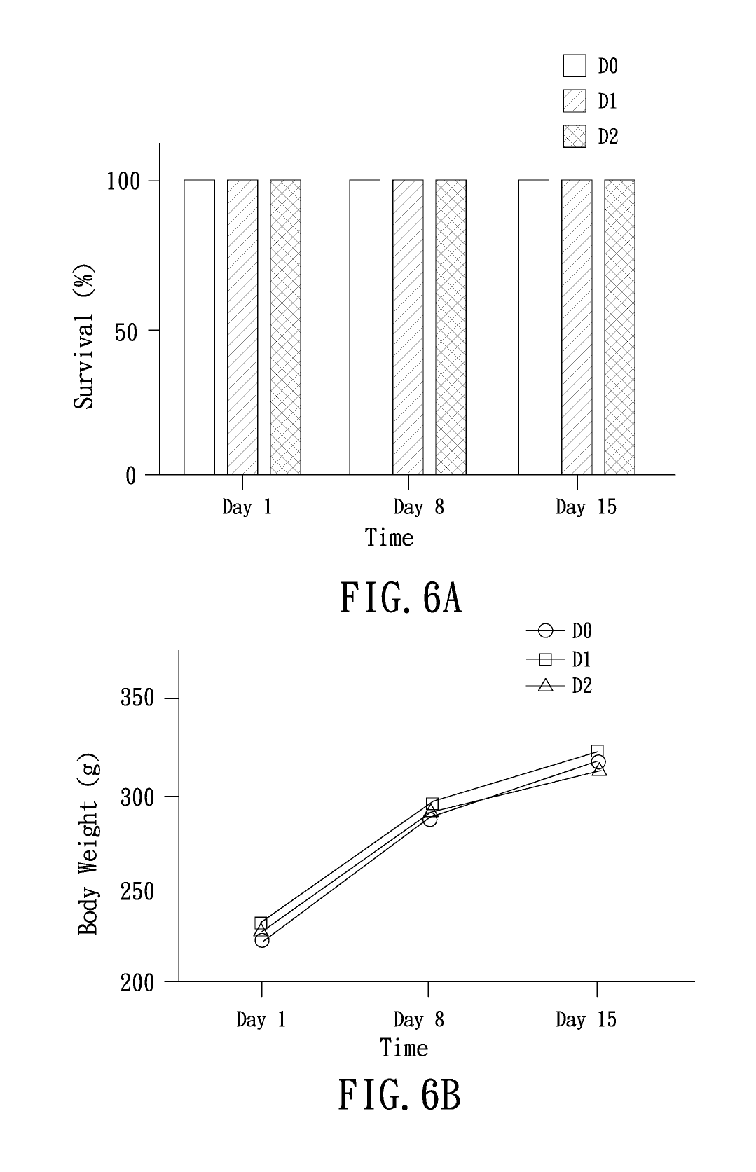

With respect to FIG. 6A, all of the SD rats of groups D1 and D2 have survived after being administered the silibinin nanoparticle of the invention for 15 days. The result shows that the silibinin nanoparticle has a safe dosage of 10 mg/kg body weight per day; namely, silibinin has a safe dosage of 5 mg/kg body weight per day. With reference to FIG. 6B, after being administered the silibinin nanoparticle of the invention for 15 days, all of the SD rats of groups D0 to D2 show a similar trend of increasing body weights. As such, the silibinin nanoparticle of the invention does not affect the normal growth of the SD rats in the safe dosage of 10 mg/kg body weight per day.

Trial (E). Bioavailability of the Silibinin Nanoparticle of the Invention

In general, when a medication is administrated to a subject, whatever the route of administration may be, the active ingredients of the medication will first enter the circulation system, then transfer into the target organ and finally act on the target organ. Although the action of the medication is affected by the difference between target organ level and blood plasma level, the elimination rate is the same. Therefore, the circulation rate and level of the medication can be explained as the factors reflecting the bioavailability.

As shown in TABLE 4, the conventional silibinin and the silibinin nanoparticle of the invention are administered to the respective groups of SD rats. After administration for 5, 15, 30, 60, 120, 360, 720, and 1440 minutes, 0.5 mL of blood samples are collected. The blood samples stand at room temperature for coagulation, followed by centrifugation to obtain serum. High-performance liquid chromatography (HPLC) is performed to analyze serum silibinin concentration.

TABLE 4

| | | Dosage (mg/kg body |
|---|---|---|
| Groups | Treatment | weight/day) |
| E1 | Silibinin | 100 |
| E2 | Silibinin Nanoparticle | 200 |
| | | (with 100 mg of |
| | | silibinin) |

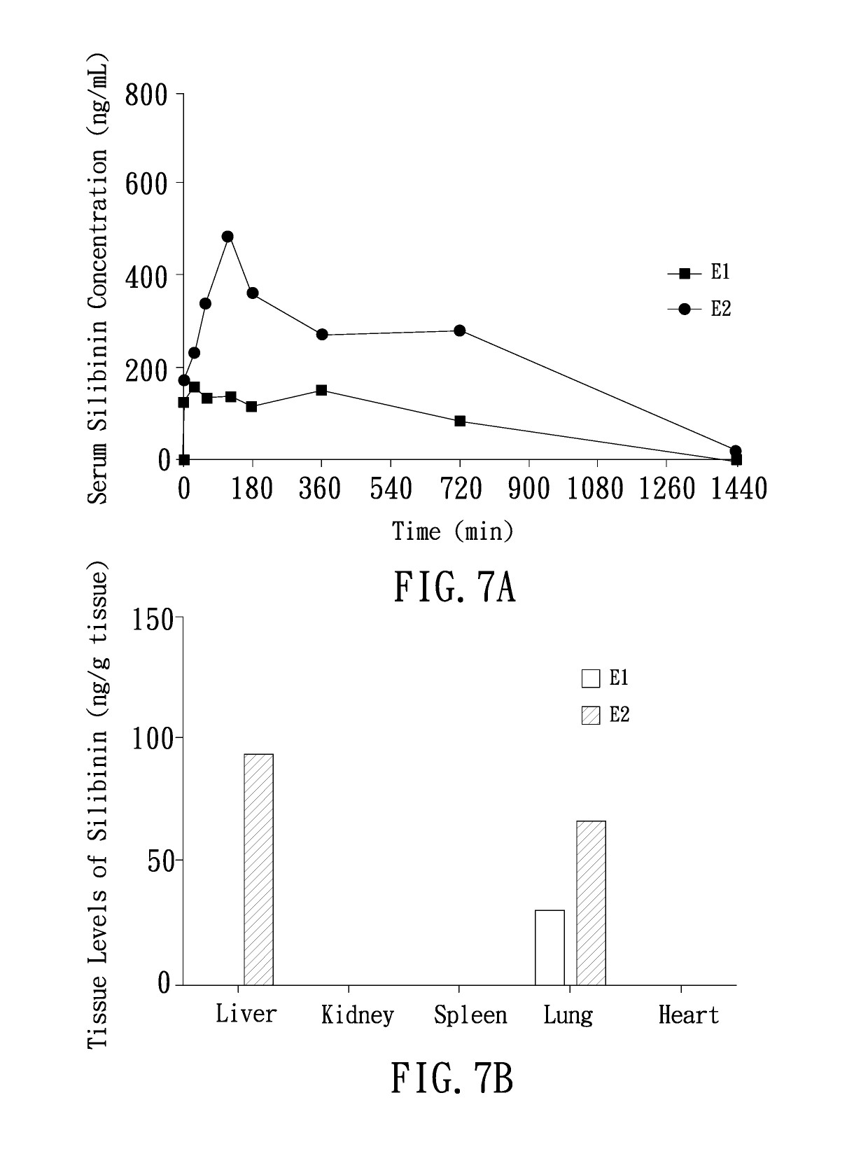

Referring to FIG. 7A, group E1 reaches the highest level of serum silibinin concentration (170 ng/mL) after administration for 15 minutes. The silibinin concentration decreases after 15 minutes and reaches 0 at 1440 minutes. That is, after 1440 minutes, the silibinin is totally eliminated from the serum of SD rats of group E1. After administration, the serum silibinin concentration of group E2 increases rapidly, reaching the highest level (500 ng/mL) after 60 minutes. As with group E1, the silibinin concentration decreases and reaches 0 at 1440 minutes. As a result, compared with the conventional silibinin, the silibinin nanoparticle of the invention has a higher serum concentration. In addition, the silibinin nanoparticle of the invention has the same elimination time as the conventional silibinin.

Furthermore, after being administered silibinin or silibinin nanoparticle for 24 hours, the SD rats of groups E1 and E2 are dissected and organs of heart, liver, spleen, lung, and kidney are collected. The collected organs are homogenized by a saline buffer, followed by extraction of silibinin with acetonitrile. The extracted silibinin is analyzed by HPLC.

With respect to FIG. 7B, in group E1, silibinin only exists in the lung, while silibinin exists in the liver and lung in group E2, with the liver silibinin concentration (90 ng/g liver tissue) being higher than the lung silibinin concentration. Accordingly, the silibinin nanoparticle of the invention can be orally administered to a subject, targeting the liver and effectively increasing the accumulated level of silibinin in the liver. That is, the silibinin nanoparticle has a better bioavailability. The silibinin nanoparticle of the invention can effectively accumulate in the liver; therefore, the silibinin nanoparticle of the invention can have improved effect on liver diseases, such as hepatitis C.

Trial (F). Effects on Anti-HCV of the Silibinin Nanoparticle of the Invention

Huh-7 cells are infected by hepatitis C virus for 3 hours. After infection, surplus hepatitis C virus are washed, followed by adding a medium with 10% fetal bovine serum (FBS) and the treatment shown in TABLE 5 for 72 hours. The resulting media is collected and the luminescence level is analyzed to evaluate HCVcc infectivity. Specifically, group F2 is treated with dimethyl sulfoxide (DMSO), group F3 is treated with interferon-α (IFN-α), a conventional medication for treating hepatitis C, group F4 is treated with PVP, group F5 is treated with the conventional silibinin dissolved in DMSO, group F6 is treated with the conventional silibinin dissolved in water, and group F7 is treated with the silibinin nanoparticle of the invention dissolved in water.

TABLE 5

| Groups | Treatment |
|---|---|
| F1 | — |
| F2 | DMSO |
| F3 | IFN-α |
| F4 | PVP |
| F5 | Silibinin/DMSO |
| F6 | Silibinin/Water |
| F7 | Silibinin Nanoparticle |

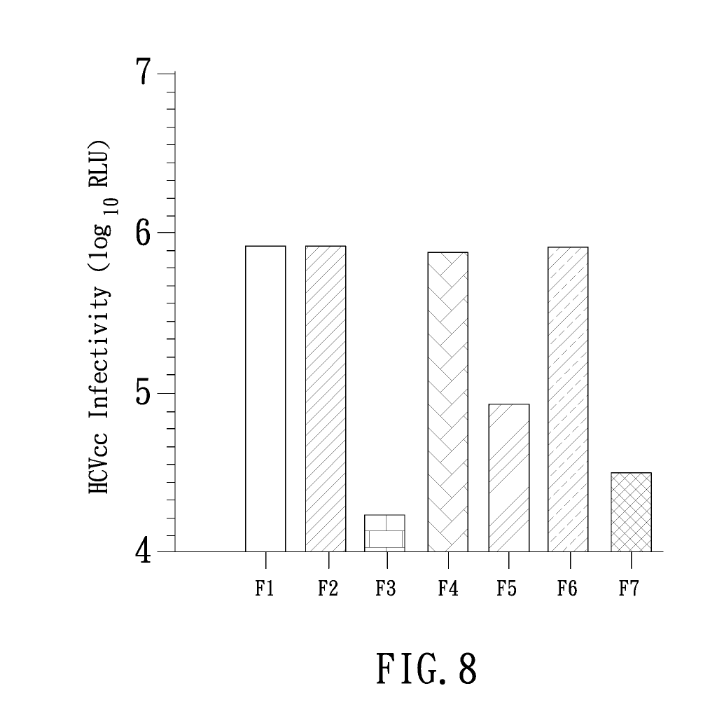

With reference to FIG. 8, groups F2 and F4 show similar results as group F1, meaning that DMSO and PVP have no effect on anti-hepatitis C. IFN-α shows HCVcc infectivity with a 4.2 log₁₀ RLU (group F3). The conventional silibinin dissolved in DMSO (group F5) shows HCVcc infectivity with a 4.9 log₁₀ RLU. However, the conventional silibinin dissolved in water (group F6) shows similar results as groups F1, F2, and F4. That is, the conventional silibinin cannot inhibit infection of hepatitis C virus due to poor water solubility. In contrast, compared with group F5, the silibinin nanoparticle of the invention (group F7) shows an improved ability to inhibit hepatitis C virus infection (4.4 log₁₀ RLU). Accordingly, compared with the conventional silibinin, the silibinin nanoparticle of the invention has an improved ability to inhibit hepatitis C virus infection.

The silibinin nanoparticle of the invention can effectively treat hepatitis C, thereby having the potential to be applied to the pharmaceutical industry as an active substance of medication or health products with anti-HCV activities. In the present invention, the silibinin nanoparticle of the invention can be given to any target individually or combined with any acceptable excipients, for example carriers or other ingredients, and is capable of being further manufactured into any form of medicament such as pill, capsule, powder, solution and pastil for easy and convenient delivery to targets.

Accordingly, the silibinin nanoparticle of the invention is in the form of a spherical structure with a particle size of 50 to 200 nm. By increasing the larger specific surface area and thus the activity of the silibinin nanoparticle, the silibinin nanoparticle of the invention has an enhanced effect on anti-HCV.

In addition, when combined with the hydrophilic polymer, the silibinin nanoparticle of the invention has improved hydrophilicity and blood solubility. The hydrophilic polymer can result in phase change of silibinin, from crystalline phase to noncrystalline phase, thereby showing improved bioavailability.

Moreover, the silibinin nanoparticle of the invention is in a patient-friendly form, easy to administer and convenient, thereby improving patient drug compliance.

Furthermore, by nanolization and noncrystallination, the silibinin nanoparticle of the invention has an improved serum silibinin concentration and effectively accumulates in the liver, thereby having a better effect for treating hepatitis C virus infection.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A silibinin nanoparticle comprising a compound silibinin and a hydrophilic polymer, wherein the silibinin nanoparticle is in a form of a spherical structure with a particle size of 50 to 200 nm.

2. The silibinin nanoparticle as claimed in claim 1, wherein the hydrophilic polymer is selected from poloxamer, polyvinyl alcohol, or polyvinylpyrrolidone.

3. The silibinin nanoparticle as claimed in claim 1, with a weight ratio of silibinin and the hydrophilic polymer being between 1:1 and 1:6.

4. The silibinin nanoparticle as claimed in claim 1, wherein the silibinin nanoparticle is manufactured as following:
dissolving silibinin in ethanol to obtain a silibinin solution;
dissolving the hydrophilic polymer in water to obtain a hydrophilic polymer solution; and
performing a nanolization reaction between the silibinin solution and the hydrophilic polymer solution to form the silibinin nanoparticle.

5. A method of treating hepatitis C, by administering the silibinin nanoparticle as claimed in claim 1 to a subject in need to treat hepatitis C thereof.

6. The method of treating hepatitis C as claimed in claim 5, wherein the silibinin nanoparticle is orally administered to the subject in need.

7. The method of treating hepatitis C as claimed in claim 5, with the silibinin nanoparticle in a dosage of 5 to 10 mg per kilogram of the subject in need for 2 weeks.
